(19) United States
(12) Patent Application Publication
(10) Pub. No.: US 2013/0022552 A1
(43) Pub. Date: Jan. 24, 2013

(54) BLOOD CLOTTING COMPOSITION AND METHOD OF USE

(71) Applicants: Clifford T. Solomon, Severna Park, MD (US); Theodore C. Solomon, Hampstead, MD (US)

(72) Inventors: Clifford T. Solomon, Severna Park, MD (US); Theodore C. Solomon, Hampstead, MD (US)

(21) Appl. No.: 13/556,158

(22) Filed: Jul. 23, 2012

Related U.S. Application Data
(60) Provisional application No. 61/510,421, filed on Jul. 21, 2011.

(51) Int. Cl. A61K 47/46; A61K 38/43; A61K 33/06; A61P 17/02; A61K 38/17; A61K 31/195; A61K 38/36; A61K 31/722; A61K 38/48; A61K 33/00

(52) U.S. Cl. 424/10.3; 424/10.1

(57) ABSTRACT

The composition is a blood clotting composition including at least one hemostatic agent and an indicator agent that adheres to the skin on or around the wound, the indicator agent being specifically identifiable to the hemostatic composition. The composition can be adapted to be applied to a wound as an emergency trauma treatment in homes, public places, trauma treatment centers, at accident locations, in the field of military combat, or any other potential site of severe trauma as a first response to reduce or stop bleeding. The composition can include at least one clotting agent, an analgesic, a bactericide (such as silver), and at least one antibiotic. An applicator device for the composition is included.

### CROSS-REFERENCE TO RELATED APPLICATION

This application claims the benefit of U.S. Provisional Patent Application Ser. No. 61/510,421, filed Jul. 21, 2011.

### BACKGROUND OF THE INVENTION

1. Field of the Invention

The present invention relates generally to the field of emergency medicine and to the field of hemostatic compositions applied by first responders for controlling bleeding, and particularly to a blood clotting composition that includes a coded indicator agent specific for the applied composition as a means of recording and communicating the use of that specific hemostatic composition for follow-on care givers.

2. Description of the Related Art

Traumatic injury is a frequent cause of morbidity and mortality worldwide. Traffic accidents account for over 40% of the trauma cases admitted at hospitals in the USA. Other accidents and crimes of violence account for another large portion of the emergency room admissions. Conflicts in conventional or unconventional warfare have become an ongoing reality around the world. In civilian communities or on the battlefield, the inability of first responders to control hemorrhage in many cases is a major contributor to the mortality rate. In combat the vast majority of the deaths occur in the field before the injured person can be transported to a treatment facility. Similarly, almost 80% of civilian trauma fatalities within the US are attributed to uncontrolled hemorrhage.

The major causes of death due to trauma are hemorrhage and neurological trauma with the rest being attributed to devastating multiple injuries, Even when the injured survive long enough to be transported to a medical facility, hemorrhage still remains the leading cause of late death and complications. Abdominal injuries pose a formidable problem, especially in young adults (age 4-8). Being the largest solid organs within the abdomen, the liver and the spleen are the most frequently injured organs and being very vascular are generally difficult organs in which to control bleeding. Massive bleeding from the liver is currently controlled by Pringle's maneuver or packing of the wound, both of which procedures require surgical intervention and cannot be applied on the battlefield or at the site of the accident. Spleen trauma can bleed profusely with minimal injury and attempts to control the bleeding are often inadequate absent a spleenectomy.

Early and effective hemorrhage control can save more lives than any other measure. All current hemostatic agents for intracavitary bleeding are designed to be used in the operating room with the cavity wide open. In an emergency situation at the site of the accident or on the battlefield uncontrolled or poorly controlled hemorrhage is often lethal. Also certain types of surgery such as laparoscopic procedures or brain surgery can be seriously complicated by internal bleeding that requires more effective and less invasive treatment.

Previous efforts have been made to find more effective ways to control bleeding and reduce the associated high mortality rates.

Over the past decade, topical application of fibrin for the purpose of initiating hemostasis as a surface coagulant has resulted in the medical community referring to such use of fibrin as that of a fibrin glue. Fibrin glue is composed of a mixture of human fibrinogen and bovine thrombin. It is sold as a kit containing separate vials of fibrinogen and thrombin solutions. These solutions are mixed together and applied to the wound in various ways, including as a paste, as a spray or on a patch. Fibrin glue, however, is an inconsistent and ineffective treatment for hemostasis. The mixing, soaking, and coating of a patch with fibrin glue requires time-consuming and cumbersome procedures during which further hemorrhaging can occur and the fibrin solutions can be washed away by intense bleeding. Examples of the continuing effort to find the right composition for fibrin glue can be found in U.S. Pat. No. 4,650,679; U.S. Pat. No. 4,377,572; U.S. Pat. No. 4,362,567; U.S. Pat. No. 5,185,001; U.S. Pat. No. 5,407,671; and U.S. Pat. No. 5,330,974, all of which are fully incorporated herein by reference.

Similarly, efforts have been made to provide an effective non-fibrin-based hemostasis treatment for topical use. Examples of such attempts to find an effective hemostatic composition include U.S. Pat. No. 6,363,551 ('551 Patent) and U.S. Pat. No. 6,454,787, both of which relate to methods for depositing thrombin in solution or powder onto a hemostatic device, such as a sponge that includes a collagen component. U.S. Pat. No. 4,752,466 ('466 Patent) is directed to a thrombin aerosol. The '466 Patent discloses thrombin delivered in a dry powdered form from a valve-actuated pressurized propellant-containing aerosol container.

Other hemostatic compositions and devices directed to the problem of providing an effective hemostatic device, such as that disclosed in the '551 Patent, which focuses on creating hemostatic fabrics that use collagen hemostatic fibers that can be pressed into a hemostatic fabric and formed to specific anatomical contours. U.S. Patent Application Publication No. 2011/0066182 discloses a four part fibrin-based, polymeric cross-linked, surgical sealant and hemostatic agent wherein the cross-linking polymers create a matrix or foundation as a means of holding the hemostatic agent in place against the site of bleeding. These and other recent efforts are evidence that the goal of providing an effective, safe hemostatic device or composition that is capable of promoting fast clotting for surface and internal hemorrhaging is an ongoing need. All of these above listed patents disclosing hemostatic compositions, devices, and methods are fully incorporated herein by reference.

Those publications listed above are a small representation of the many and varied efforts that have been made in the past to provide a fast, effective hemostatic device or composition capable of field or emergency room use in the overall effort to save lives by controlling bleeding from injuries and wounds. Among the many possible hemostatic agents that are included in such compositions and products are: thrombin, fibrinogen, collagen, calcium ions, Arginine-Glycine-Aspartic Acid (RGD) peptide, protamine sulfate, epsilon amino caproic acid, chitin, and others.

In addition, many of the compositions also include varying amounts and combinations of additional therapeutic agents and medicaments, such as agents that promote wound healing and or reduce pain. Agents that promote wound healing and or reduce pain include anti-inflammatory agents (steroidal and non-steroidal), such as agents that inhibit leukocyte migration into the area of surgical injury, anti-histamines, agents that inhibit free radical formation, and bacteriostatic or bacteriocidal agents.

As discussed above there are numerous widely varied hemostatic compositions in use. However, these conventional compositions share a common deficiency in their purpose to control bleeding and save lives. That shared deficiency is a lack of communication to follow-on medical/surgical care providers of precisely what compounds, medicaments, agents, etc. were included in the hemostatic product that was administered to the patient on the battlefield, at the accident site, in the home, or in the emergency room by the first responders. Ignorance of precisely what prior medications were administered to trauma patients by first responders is critically important to ensure that follow-on providers do not administer unnecessary or even dangerous additional drugs to the patient. Without a precise knowledge of what medicinal drugs and therapeutic compounds the patient has been previously given, the follow-on provider can very easily provide the patient with duplicate doses, incompatible medicaments, counter-active drugs, and other ill-advised treatments that may endanger the life of the patient.

Additionally, hemostatic compositions can include ingredients to which the trauma victim may be allergic. Follow-on providers observing a trauma patient in distress can be assisted in their efforts to discover the source of the distress if they are provided with a full knowledge of all drugs, compounds, or other ingredients of the hemostatic composition that was earlier applied to the victim. Given such information it is possible that allergic reactions, overdoses or other sources of physiological distress can be more quickly recognized and addressed. The follow-on surgical and medical care providers should be quickly, clearly, and accurately informed of precisely what therapeutics, analgesics, or other agents have been administered to the patient through the emergency treatment of the wound within the hemostatic composition. Current reliance on the contents labels provided on or in the hemostatic product container is only useful if the contents label remains with the trauma victim as he is transferred to one or more follow-on care facilities. Alternatively, providing a written first responder record of the emergency treatment provided to the trauma victim may or may not include the fact that a hemostatic composition was earlier administered to the wound. Further, even if that record includes the history of administering a hemostatic composition, it probably would not include a detail listing of the all of the chemical components of the applied composition. A first provider medical record is also subject to being separated or lost during transport of the patient to follow-on providers.

It is therefore of critical importance for the safety of the trauma patient to whom a hemostatic device or composition has been administered by first providers to include a device for accurately and reliably informing follow-on medical/surgical providers of precisely what components were included in that hemostatic composition. Provision of an effective hemostatic composition that includes this critically important information with the patient is an issue of patient safety that has not thus far been addressed by the medical profession. It is a much-needed advancement in the field of emergency medicine and the surgical arts.

Thus, a blood clotting composition and method of use solving the aforementioned problems is desired.

### SUMMARY OF THE INVENTION

The blood clotting composition is primarily intended for use by first responders to emergency situations wherein injured persons are suffering a serious degree of uncontrolled bleeding and, absent the application of immediate hemostatic measures, might succumb to their injuries. The composition is a blood clotting composition including at least one hemostatic agent and an indicator agent that adheres to the skin on or around the wound, the indicator agent being specifically identifiable to the hemostatic composition. The composition is adapted to be applied to a wound as an emergency trauma treatment in homes, public places, trauma treatment centers, at accident locations, in the field of military combat, or any other potential site of severe trauma as a first response to reduce or stop bleeding. The composition can include at least one clotting agent, an analgesic, a bactericide (such as silver, sulfur, or the like), and at least one antibiotic.

One or more of the components of the therapeutic composition can be provided in nanoparticle form and a portion or all of the varied components of the composition can be encapsulated in a time release microparticle matrix having varying programmed degrees of delayed release when applied to or inserted into a wound of a patient.

The composition includes a coded indicator agent that upon contact with the wound provides a marker on or around the wound. Preferably, this marker is a biocompatible color marker that is visibly distinguishable from any physiologically generated color, and therefore easily recognizable when present on the skin. It is, however, possible that a skin marker can be applied as a component of the hemostatic composition that is not within the visible light spectrum, but requires a non-visible light source to be seen. This recognizable color indicator provides an advice/warning to follow-on treatment medical personnel that the wound has been treated with the blood clotting composition. The composition can be provided in a preloaded wound treatment device, such as a syringe-type applicator, a mechanical gun device, a composition impregnated wound covering, such as an adhesive bandage or a compression bandage, a packaged powder, spray, aerosol, foam or gel that can be directly applied to the wound. Other suitable direct applicators can also be used to allow rescue personal, emergency medical personnel, family members, or others with little or no professional medical training to apply the composition to wounds in emergency situations and, due to the color indicator, be able to passively communicate the fact of the application to follow-on providers. The blood clotting composition is particularly useful for medical emergency first responders, police, and military personnel to stop bleeding from wounds on site at the location where the injury occurred. All applicators used as part of the invention include information clearly labeled on the applicator or on the composition impregnated wound dressing that provides an advice/warning to medical personnel listing all components of the composition that was applied to or into the wound. In addition to package/applicator labeling the precise composition of each variant of the hemostatic composition can be listed under its specific color-code as information for follow-on providers in print or on-line in the Physician's Desk Reference (PDR), Poison Control Center, or other on-line data bases that are easily and immediately accessible to follow-on medical or surgical care providers.

These and other features of the present invention will become readily apparent upon further review of the following specification and drawings.

### BRIEF DESCRIPTION OF THE DRAWINGS

FIG. 1A is a bottom view of an exemplary adhesive bandage having an adhesive section for holding a gauze section against a wound, the gauze section containing a blood clotting composition according to the present invention.

FIG. 1B depicts an example of a compression bandage having a centrally disposed gauze section capable of containing a blood clotting composition according to the present invention, the gauze section being attached to an adhesive section that fully surrounds the gauze section and serves to secure the bandage against a wound.

FIG. 2 depicts an exemplary syringe useful for rapid delivery of a blood clotting composition according to the present invention into a wound.

FIG. 3A depicts an exemplary application gun useful for rapid delivery of a blood clotting composition according to the present invention deep into a wound using a detachable delivery extension attachment.

FIG. 3B depicts an exemplary composition spreader attachment that, in place of the extension attachment shown in FIG. 3A, can be selectively attached to the application gun shown in FIG. 3A and used to facilitate rapid delivery of a blood clotting composition according to the present invention over a broad surface area of a wound.

Similar reference characters denote corresponding features consistently throughout the attached drawings.

### DETAILED DESCRIPTION OF THE PREFERRED EMBODIMENTS

The blood clotting composition includes at least one blood clotting agent that can be applied to a wound as an emergency trauma treatment to accelerate blood clotting and stem the loss of blood, thereby preventing death by exsanguination. Provided herein are descriptions of various examples of blood clotting compositions, also referred to as herein as hemostatic compositions, and possible composition delivery devices, which are generally shown at 10. Also described below are composition components and a system of use that provides the ability for the follow-on medical or surgical care givers to identify the precise components included in the hemostatic composition, which was earlier administered by the first providers to the trauma patient. The examples of these compositions and delivery devices, which are generally shown at 10 and described below, are discussed in detail with the understanding that the teachings thereof can be applied to virtually all hemostatic compositions as required.

The blood clotting composition is primarily intended for use by first responders to emergency situations wherein injured persons are suffering a serious degree of uncontrolled bleeding and, absent the application of immediate hemostatic measures, might succumb to their injuries. The composition can include at least one blood clotting agent, an analgesic, and an antibiotic therapeutic and/or other components that can be applied to a wound as an emergency trauma treatment in homes, public places, trauma treatment centers, at accident locations, in the field of military combat, or any other potential site of severe trauma as a first response to reduce or stop bleeding. Embodiments of the composition can include at least one clotting agent, an analgesic, a bactericide such as, for example, silver and/or at least one antibiotic as well as other therapeutic ingredients, preservatives, stabilizers, foundation forming polymers, fillers, and the like.

One or more of the embodiments of the hemostatic composition can be provided wherein some of the components are provided in nanoparticle form and a portion or all of the varied components of the composition can be encapsulated in a time release microparticle matrix having varying programmed degrees of delayed release when applied to or inserted into a wound of a patient. The use of biodegradable polymers to form microencapsulated matrices of compositions for treating hemorrhagic conditions is disclosed in U.S. Patent Application Publication No. 2011/0142937, the complete disclosure of which is incorporated herein by reference.

The hemostatic composition includes a coded indicator agent that upon contact with the wound provides a colored biocompatible marker that temporarily adheres to the skin adjacent the wound and is distinguishable from any physiologically generated color. Any known non-toxic, biocompatible stain, dye, ink, or other non-permanent coloring agent can be employed in the composition as a composition identifying indicator for the hemostatic composition. This visual marker is easily recognized in and around the tissue at the wound site for purpose of indicating which specific hemostatic composition has been applied to or into the wound. This recognizable color indicator provides a much needed advisory warning for follow-on treatment medical personnel that the wound has been treated with a particular composition having specific chemical/medicinal components.

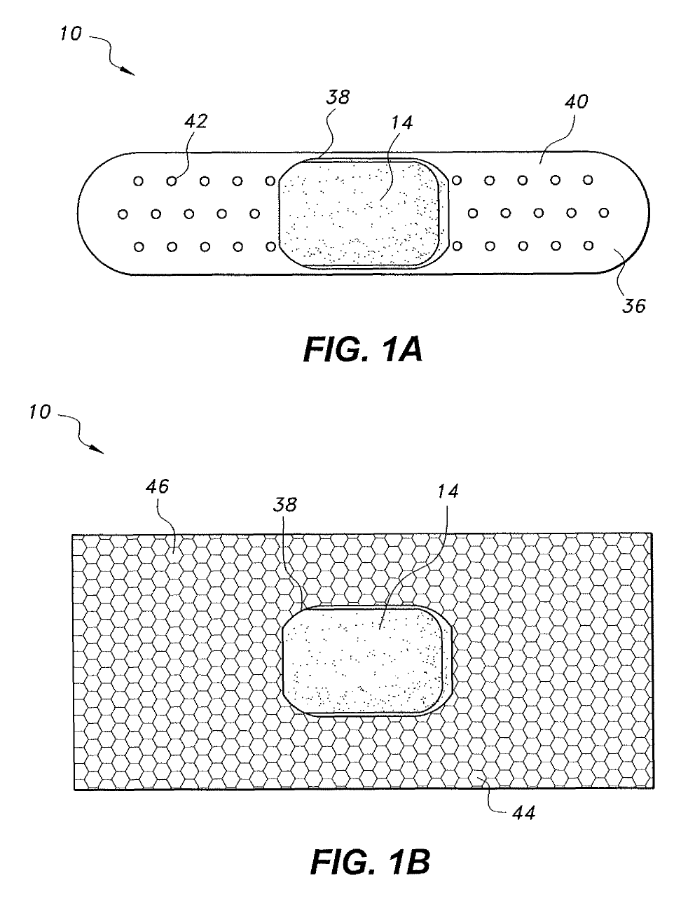

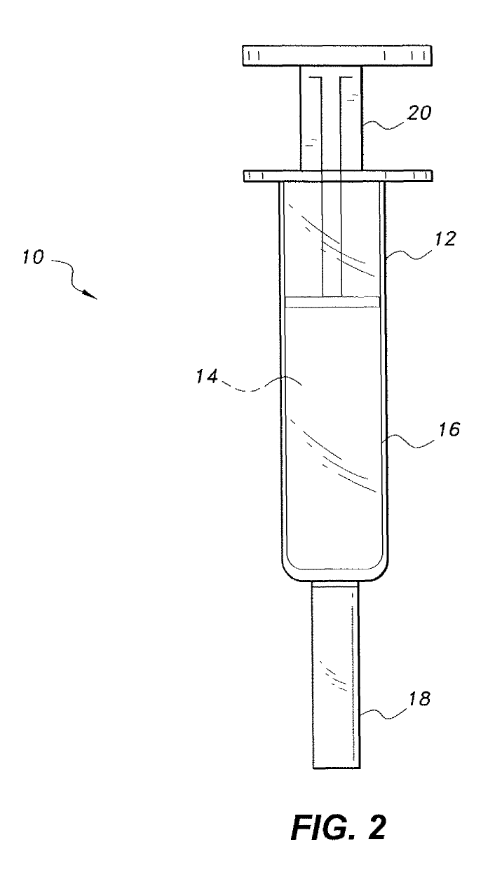

As shown in FIGS. 1A-3B, the composition can be provided in different embodiments of a preloaded wound treatment device 10. Examples of available application devices 10 include, for example, a syringe-type applicator 12 as shown in FIG. 2. The hemostatic composition 14 can be provided preloaded within the barrel 16 of the syringe 12 and, when needed, expelled from the expulsion stem 18 by the manual depression of the syringe plunger 20. If any embodiments of the hemostatic composition should not be premixed prior to application to a wound, a multi-barrel syringe can be provided. An example of a multi-barrel syringe is disclosed in U.S. Pat. No. 6,116,900, the disclosure of which is fully incorporated by reference.

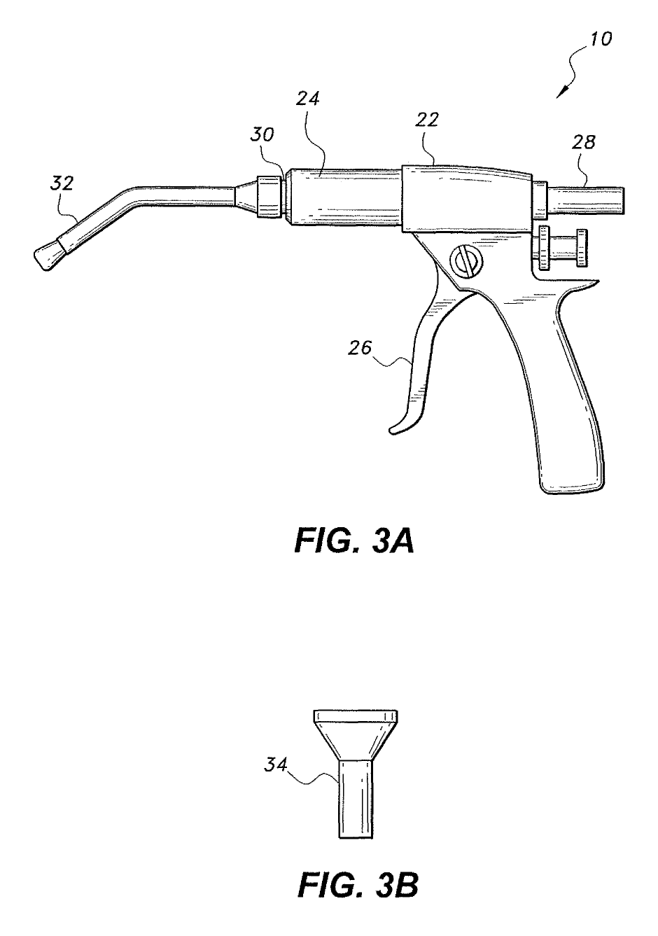

The device 10 can also be provided in the form of a mechanical gun 22 with the composition 14 preloaded within the gun chamber 24. Upon manual actuation of the gun trigger mechanism 26, the piston forcibly ejects the composition 14 from the distal end 30 of the gun chamber 24. As shown in FIG. 3A, the gun 22 can be fitted with a removable applicator extension 32, which facilitates application of the composition 14 into deep wounds. As shown in FIG. 3B, a composition spreader attachment 34, which can be used in place of the applicator extension 32 when it is preferable to apply the composition 14 onto a broad surface wound.

Another example of the device 10, which is useful for application of the composition 14 to a surface wound, is an adhesive bandage 36 that includes a gauze-like matrix 38 having the capacity to carry a preloaded effective amount of the composition 14, which, upon application to the wound, can be gradually released onto the damaged portion of the skin and cause a hemostatic effect on the wound. The adhesive bandage is held against the skin by an adhesive backing 40. The adhesive backing can be provided with multiple pores 42 to allow the underlying skin to which it is attached access to ambient air.

An additional example of the device 10, which is useful for application of the composition 14 to a surface wound with excessive bleeding, is a compression bandage 44. Similar to the adhesive bandage 36, the compression bandage 44 includes a gauze-like matrix 38 having the capacity to carry a preloaded effective amount of the composition 14, which upon application to the wound can be gradually released on to the damage portion of the skin and cause a hemostatic effect on the wound. Unlike the adhesive bandage 36, the compression bandage 44 is held against the skin by an airtight adhesive section 46 that fully surrounds the gauze section and is devoid of any pores or other openings that would permit leakage of the composition 14 or passage of air, blood, or other fluids through the seal created by the adhesive section 46.

The adhesive bandage 36 or compression bandage 44 can be configured in any shape or size, as needed. The bandages 36, 44 will preferably be flexible so as to be able to follow the contour of the body surface and provide full contact with the wound and surrounding area. Preferably, the composition 14 provided with the bandages, 36, 44 is the form of a dry power, gel or porous microsphere.

The hemostatic composition can also be provided as a packaged powder, spray, aerosol, foam or gel that can be directly applied to the wound. Other suitable applicators can also be provided to allow rescue personal, emergency medical personnel, family members, or others with little or no professional medical training to apply the composition to wounds in emergency situations. Due to the included composition specific color agent and its obvious visual presence on the skin of the patient, the application of the specific hemostatic composition with its particular components can be, by visual observation alone, communicated to follow-on providers. The blood clotting composition is particularly useful for medical emergency first responders, police, and military personnel to stop bleeding from wounds in the field. All applicators used as part of the invention include information clearly labeled on the applicator or on the composition impregnated wound dressing as an advice/warning to medical personnel listing the components of the composition applied to or into the wound. Additionally, the precise composition of each embodiment of the hemostatic composition can be listed with its specific color-code as information for follow-on providers in the Physician's Desk Reference (PDR), Poison Control Center, or any other print or on-line databases that are easily and immediately accessible. The use of such hemostatic compositions provides a previously unavailable communicative composition that enables the follow-on provider to continue treatment and medication of the patient without creating additional hazards by treating and medicating a trauma patient without knowledge of what compositions and medicaments have been earlier provided. Importantly, any hemostatic composition when formulated as described herein will include a passive communication device that enables follow-on care providers to treat the trauma patient without fear of administering dangerous overdoses unintentional and potentially dangerous admixtures of earlier provided medication included in the hemostatic composition applied by the first providers. This advantageous feature of the composition does not rely on the need to provide any written emergency treatment medical records or the like.

As indicated above, the composition can include at least one hemostatic agent. Suitable hemostatic agents in effective amounts for stimulating or facilitating hemostasis, include, but are not limited to: thrombin, an enzyme which converts fibrinogen to fibrin; calcium, sodium, magnesium or other ions that stimulate hemostasis; protamine sulfate, an epsilon amino caproic acid, fibrinogen, and chitin.

Thrombin is a hemostatic active ingredient suitable for use in hemostatic compositions and is frequently included with compositions containing fibrin, which is enzymatically converted to fibrinogen, as disclosed in U.S. Pat. No. 6,361,551, the complete disclosure of which is fully incorporated herein by reference. A thrombin stabilizer can be included in the composition such as, for example a thrombin stabilizing effective amount of sugar (such a mannitol, sorbitol, glucose and sucrose), a thrombin inhibitor, a calcium chelator, and a protein. These protein agents are also often included in compositions and devices containing collagen or collagen fibrils, which also have a hemostatic activity and have been employed in hemostatic compositions having wet, dry, powdered, or foam character for application to a patient's wound. Exemplary methods of preparation and use for hemostatic compositions, which are formulated around the use of thrombin and collagen as a compounds supporting hemostasis, are disclosed in U.S. Pat. No. 6,454,787 and U.S. Pat. No. 6,096,309, which are herein entirely incorporated by reference.

The hemostatic composition, which typically will be applied to a wound by a first responder at the incident site can include as prophylactic antibiotic selected from antibiotic classes including: penicillins, penicillin combinations, sulfonamides, lincosamides, carbapenems, tetracyclines, aminoglycosides, as well as other suitable antibiotic compositions and combinations thereof.

Other possible components of a hemostatic composition include hormonal agents, such as growth factors to promote wound healing and other therapeutic agents. In some embodiments of a hemostatic composition a wound-sealant composition, as described in U.S. Patent Application Publication No 2009/0148502 and including at least a cross-bridging binding agent of silica nanoparticles having potential reactive surface hydroxyl groups and possibly additional components including a fluid removal agent, an adhesive clumping agent, a swelling agent, a drug delivery vehicle such as a nanoparticle or microparticle, a clot enhancing composition, and an activator or accelerator, the complete disclosure of which is fully incorporated herein by reference.

The hemostatic composition can also contain adjuvants including preservative agents, wetting agents, emulsifying agents and dispersing agents, additional antibiotics alone or in combination with antifungal agents, for example, parabens, chlorobutanol, phenol, sorbic acid, and the like. It is also possible to include isotonic agents such as sugars, sodium chloride and the like. Additionally, agents for delaying absorption, such as aluminum monostearate and gelatin, can also be included in the composition as desired.

It is to be understood that the present invention is not limited to the embodiments described above, but encompasses any and all embodiments within the scope of the following claims.

We claim:

1. A blood clotting composition, comprising:
at least one hemostatic agent for application to a wound; and
an indicator agent adhering to the skin around the wound to which the composition is applied, the indicator agent having a property uniquely identifying the hemostatic composition.

2. The blood clotting composition of claim 1, wherein said property uniquely identifying the hemostatic composition is a specific color.

3. The blood clotting composition according to claim 1, wherein said indicator agent is in the visible light spectrum.

4. The blood clotting composition according to claim 1, wherein said indicator agent is a non-toxic biocompatible color-coded indicator selected from the group consisting of a stain, a dye, and an ink.

5. The blood clotting composition according to claim 1, wherein said hemostatic agent contains at least one clotting agent selected from the group consisting of thrombin, an enzyme that converts fibrinogen to fibrin; calcium, sodium, magnesium; protamine sulfate, an epsilon amino caproic acid, fibrinogen, and chitin.

6. The blood clotting composition according to claim 1, wherein said hemostatic agent includes a time release polymer.

7. The blood clotting composition according to claim 1, further comprising an analgesic agent.

8. The blood clotting composition according to claim 1, further comprising an antibiotic agent.

9. The blood clotting composition according to claim 1, further comprising a hormonal growth factor.

10. The blood clotting composition according to claim 1, further comprising a preservative,

11. A method of treating a bleeding wound on a subject, the method comprising the steps of:
providing a hemostatic composition, the composition having:
at least one hemostatic agent for application to a wound; and
an indicator agent adhering to the skin around the wound to which the hemostatic composition is applied, the indicator agent having a property uniquely identifying the hemostatic composition;
applying the hemostatic composition to the wound;
identifying the indicator agent and correlating the identified indicator agent to a specific hemostatic composition using a listing of hemostatic compositions, the listing including identification of components of each of the listed hemostatic compositions.

12. The method of treating a bleeding wound according to claim 11, wherein said listing of hemostatic compositions is provided in a recorded form selected from the group consisting of printed information, electronic retrievable information, and telephonically available information.

13. The method of treating a bleeding wound according to claim 11, wherein said indicator agent is a color-coding agent, the method further comprising the step of identifying active ingredients in the hemostatic composition by identification of a color stain in and around the clotting wound.

14. A kit for use in treating a bleeding wound on a subject, the kit comprising:
a hemostatic composition, the composition having:
at least one hemostatic agent for application to a wound; and
an indicator agent adhering to the skin around the wound to which the hemostatic composition is applied, the indicator agent having a property uniquely identifying the hemostatic composition; and
an application device adapted to facilitate application of the hemostatic composition onto or into the wound.

15. The kit for use in treating a bleeding wound according to claim 15, wherein said indicator agent comprises a non-toxic biocompatible color-coded indicator selected from the group consisting of a stain, a dye, and an ink.

16. The kit for use in treating a bleeding wound according to claim 15, wherein said application device comprises a syringe.

17. The kit for use in treating a bleeding wound according to claim 15, wherein said application device comprises a mechanical gun.

18. The kit for use in treating a bleeding wound according to claim 15, wherein said application device comprises an adhesive bandage including gauze, the gauze being prepackaged with the hemostatic composition adsorbed thereon.

19. The kit for use in treating a bleeding wound according to claim 15, wherein said application device comprises a compressive bandage including gauze, the gauze being prepackaged with the hemostatic composition adsorbed thereon.

20. The kit for use in treating a bleeding wound according to claim 15, wherein said indicator agent comprises a non-toxic biocompatible color-coded indicator selected from the group consisting of a stain, a dye, and an ink, the kit further comprising a label identifying the active agents in the hemostatic agent according to the color of the indicator agent.
